(19) United States
(12) Patent Application Publication
(10) Pub. No.: US 2025/0385112 A1
(43) Pub. Date: Dec. 18, 2025

(54) SEMICONDUCTOR MANUFACTURING EQUIPMENT

(71) Applicant: SAMSUNG ELECTRONICS CO., LTD., Suwon-si (KR)

(72) Inventors: MINHYOUNG KIM, Suwon-si (KR); Hyunho Doh, Suwon-si (KR); SUNG YONG PARK, Suwon-si (KR); Seokjin Lee, Suwon-si (KR); Woorim Lee, Suwon-si (KR); Junhyeok Choi, Suwon-si (KR)

(73) Assignee: SAMSUNG ELECTRONICS CO., LTD., Suwon-si (KR)

(21) Appl. No.: 19/086,879

(22) Filed: Mar. 21, 2025

(30) Foreign Application Priority Data
Jun. 12, 2024 (KR) 10-2024-0076294

(51) Int. Cl. H01L 21/67; G01N 21/73; G01N 33/00; H01J 37/32

(52) U.S. Cl. CPC H01L 21/67253; G01N 21/73; G01N 33/0073; H01J 37/32357; H01J 37/32422; H01J 37/32834; H01L 21/67017; H01J 2237/2445; H01J 2237/24585

(57) ABSTRACT

Disclosed is semiconductor manufacturing equipment. The semiconductor manufacturing equipment includes: a remote plasma source used configured to generate plasma; a reaction chamber coupled to the remote plasma source, and defining a space in which a wafer is processed; an exhaust pipeline coupled to the reaction chamber through an exhaust port, and configured to discharge by-products generated in the reaction chamber; a first pipeline coupled to the reaction chamber; a second pipeline coupled to the exhaust pipeline through the exhaust port; and an analyzer configured to analyze a first gas mixture received from the reaction chamber through the first pipeline.

### CROSS-REFERENCE TO RELATED APPLICATION

This application claims priority to Korean Patent Application No. 10-2024-0076294, filed on Jun. 12, 2024, in the Korean Intellectual Property Office, the disclosure of which is incorporated by reference herein in its entirety.

### BACKGROUND

The present disclosure relates to semiconductor manufacturing equipment.

A series of processes, such as deposition, etching, and cleaning, may be performed to manufacture semiconductor elements. These processes may be performed through a deposition device, an etching device, or a cleaning device provided with process chambers. Plasma technologies, such as capacitive coupled plasma (CCP), inductive coupled plasma (ICP), or a combination of CCP and ICP are being adopted to improve selectivity, change properties of films, and minimize damage to the films. Plasma technologies include a direct plasma technology of directly generating plasma in a process chamber that is a wafer processing space, and a remote plasma technology of generating plasma outside the process chamber and supplying it to the process chamber.

### SUMMARY

One or more embodiments provide semiconductor manufacturing equipment that may support real-time monitoring of a reaction area in a reaction chamber.

According to an aspect of an embodiment, semiconductor manufacturing equipment includes: a remote plasma source used configured to generate plasma; a reaction chamber coupled to the remote plasma source, and defining a space in which a wafer is processed; an exhaust pipeline coupled to the reaction chamber through an exhaust port, and configured to discharge by-products generated in the reaction chamber; a first pipeline coupled to the reaction chamber; a second pipeline coupled to the exhaust pipeline through the exhaust port; and an analyzer configured to analyze a first gas mixture received from the reaction chamber through the first pipeline.

According to another aspect of an embodiment, semiconductor manufacturing equipment includes: a remote plasma source configured to generate plasma; a reaction chamber coupled to the remote plasma source, and defining a space in which a wafer is processed; a shower head located between the remote plasma source and the reaction chamber; an exhaust pipeline coupled to the reaction chamber through an exhaust port, and configured to discharge by-products generated in the reaction chamber; a first pipeline coupled to the reaction chamber; a first manual valve configured to attach the first pipeline to the reaction chamber; a needle valve in the first pipeline, wherein the needle valve is configured to control a flow rate of a gas mixture flowing in the first pipeline; a second pipeline coupled to the exhaust pipeline through the exhaust port; a second manual valve in the second pipeline, and used for mounting or demounting the second pipeline on or from the exhaust port; a throttle valve in the second pipeline, wherein the throttle valve is configured to control a flow rate of the gas mixture flowing in the second pipeline; an analyzer configured to analyze the gas mixture received from the reaction chamber through the first pipeline; and an on-off valve in the first pipeline, wherein the on-off valve is configured to selectively allow flow of the gas mixture to the analyzer.

According to another aspect of an embodiment, semiconductor manufacturing equipment includes: a remote plasma source configured to generate plasma; a reaction chamber coupled to the remote plasma source, and defining a space in which a wafer is processed; an exhaust pipeline coupled to the reaction chamber through an exhaust port, wherein the exhaust pipeline is configured to discharge by-products generated in the reaction chamber; an analyzer coupled to the reaction chamber through a first pipeline, wherein the analyzer is coupled to the exhaust pipeline through a second pipeline, and is configured to analyze a gas mixture received from the reaction chamber; and an equipment controller configured to control at least one of a parameter for the remote plasma source or a parameter for the reaction chamber, based on a monitoring result received from the analyzer.

### BRIEF DESCRIPTION OF DRAWINGS

The above and other objects and features will become apparent by describing in detail embodiments thereof with reference to the accompanying drawings.

FIG. 1 is a view illustrating semiconductor manufacturing equipment according to an embodiment;

FIG. 2 is a flowchart illustrating an example of a real-time monitoring operation for a reaction area and a control factor setting operation according to an embodiment;

FIG. 3 is a flowchart illustrating an example of an operation for selecting a control factor according to an embodiment;

FIGS. 4A and 4B illustrate examples of monitoring results for an etch component and a passivation component when gas “X” is introduced as a source gas according to an embodiment;

FIGS. 5A and 5B illustrate examples of monitoring results for an etch component and a passivation component when gas “Y” is introduced as a source gas according to an embodiment;

FIGS. 6A and 6B illustrate examples of monitoring results for an etch component and a passivation component when gas “Z” is introduced as a source gas according to an embodiment;

FIGS. 7A and 7B illustrate an example of setting a control range when gas “Y” is selected as a control factor according to an embodiment;

FIG. 8A and FIG. 8B illustrate an example of setting a control ranges when gas “Z” is selected as a control factor according to an embodiment;

FIG. 9 is a view illustrating semiconductor manufacturing equipment according to an embodiment;

FIG. 10 is a view illustrating semiconductor manufacturing equipment according to an embodiment;

FIG. 11 illustrates an example of operation of a semiconductor manufacturing equipment, for selecting a monitoring target area according to an embodiment;

FIG. 12 is a view illustrating semiconductor manufacturing equipment according to an embodiment; and

FIG. 13 is a view illustrating semiconductor manufacturing equipment according to an embodiment.

### DETAILED DESCRIPTION

Hereinafter, embodiments are described in detail with reference to the accompanying drawings. Like components are denoted by like reference numerals throughout the specification, and repeated descriptions thereof are omitted. It will be understood that when an element or layer is referred to as being “on,” “connected to” or “coupled to” another element or layer, it can be directly on, connected or coupled to the other element or layer, or intervening elements or layers may be present. By contrast, when an element is referred to as being “directly on,” “directly connected to” or “directly coupled to” another element or layer, there are no intervening elements or layers present. Embodiments described herein are example embodiments, and thus, the present disclosure is not limited thereto, and may be realized in various other forms. Each embodiment provided in the following description is not excluded from being associated with one or more features of another example or another embodiment also provided herein or not provided herein but consistent with the present disclosure.

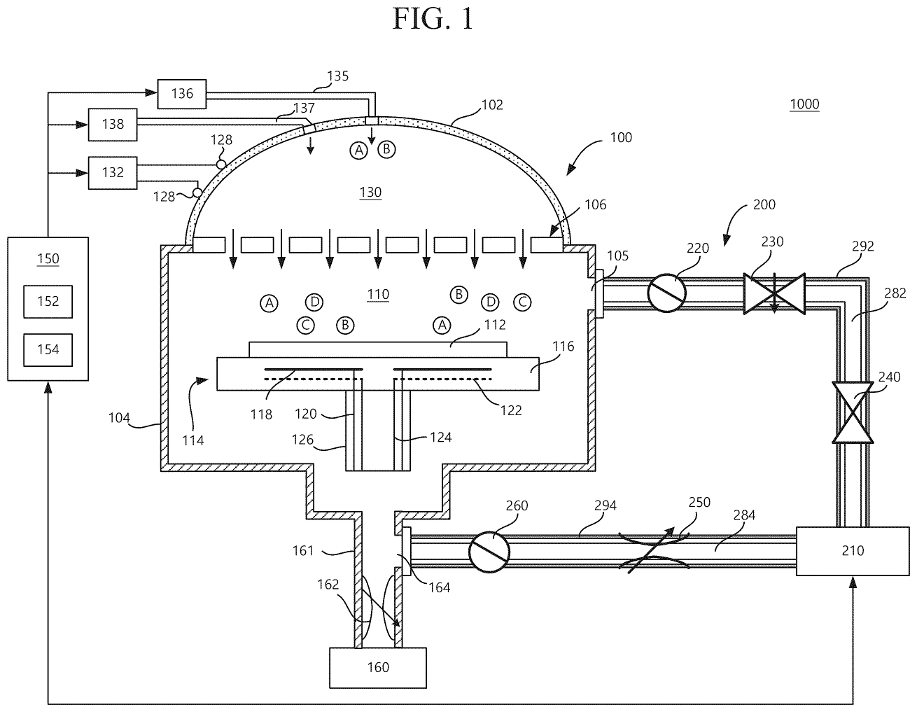

FIG. 1 is a view illustrating semiconductor manufacturing equipment according to an embodiment.

Semiconductor manufacturing equipment 1000 according to an embodiment may include a plasma processing apparatus 100 and a reaction area monitoring apparatus 200. The reaction area monitoring apparatus 200 supports a real-time monitoring operation for a reaction area 110 in the plasma processing apparatus 100, and the plasma processing apparatus 100 may control related parameters and/or conditions to implement required performance and process results based on the monitoring results. Accordingly, the semiconductor manufacturing equipment 1000 may efficiently find parameters and/or conditions for an optimal process or facility to implement required performance and process results.

Referring to FIG. 1, the plasma processing apparatus 100 may include a remote plasma source 102, a reaction chamber 104, a wafer stage 114, and an exhaust part 160.

The remote plasma source 102 may be coupled to the reaction chamber 104. For example, the remote plasma source 102 may be fluidly coupled to the reaction chamber 104 through a shower head 106. Here, “fluidly coupled” means “coupled so that a fluid may flow”, and may include indirect coupling as well as direct coupling. For example, a third component may be disposed between the first and second components that are fluidly coupled to each other.

In an embodiment, the remote plasma source 102 may generate plasma in a plasma area 130 through an inductively coupled plasma (ICP) method. However, this is provided as an example, and according to an embodiment, the remote plasma source 102 may also generate plasma in the plasma area 130 through a capacitively coupled plasma (CCP) method, a microwave method, and the like.

The shower head 106 may be located between the remote plasma source 102 and the reaction chamber 104. According to an embodiment, the shower head 106 may include an ion filter for filtering ions to limit ion impact damage to a wafer 112. For example, when radicals and/or ions are generated in the remote plasma source 102, the ions may be filtered by the shower head 106, and the radicals may be supplied to the reaction chamber 104.

The reaction chamber 104 may provide a sealed space for performing deposition, etching, and cleaning processes on the wafer 112. The space in the reaction chamber 104, in which the deposition, etching, and cleaning processes are performed, may be referred to as a reaction area 110. For example, the reaction chamber 104 may include a metal, such as aluminum or stainless steel.

A wafer stage 114 for supporting the wafer 112 may be disposed in an interior of the reaction chamber 104. For example, the wafer stage 114 may serve as a susceptor for supporting the wafer 112.

The wafer stage 114 may include an electrostatic chuck 116 for maintaining the wafer 112 on an upper side thereof an electrostatic suction force. For example, the electrostatic chuck 116 may include at least one electrostatic clamping electrode 118 that is embedded in a body of the electrostatic chuck 116.

In an embodiment, at least two of the electrostatic clamping electrodes 118 may be on the same plane or substantially on the same plane. For example, each of the electrostatic clamping electrodes 118 may be on the same plane or substantially on the same plane. The electrostatic clamping electrodes 118 may be supplied with electric power by a DC power source or a DC chucking voltage such that the wafer 112 may be maintained on the electrostatic chuck 116 by an electrostatic suction force. In an embodiment, the electric power to the electrostatic clamping electrode 118 may be provided through a first electric line 120.

The electrostatic chuck 116 may further include at least one heating element 122 that is embedded in a body of the electrostatic chuck 116. For example, the at least one heating element 122 may include a resistance heater. In an embodiment, the at least one heating element 122 may be disposed under the at least one electrostatic clamping electrode 118. However, according to an embodiment, at least one heating element 122 may be disposed on an upper side of at least one electrostatic clamping electrode 118.

The at least one heating element 122 may be configured to heat the wafer 112. For example, the at least one heating element 122 may provide a selective temperature control to the wafer 112. According to an embodiment, the electric power may be provided to the at least one heating element 122 through a second electric line 124.

The wafer stage 114 may further include a stem 126 that is coupled to a lower side of the electrostatic chuck 116. The stem 126 may function as a column that supports the electrostatic chuck 116. According to an embodiment, the stem 126 may have through-holes, in which the first electric line 120 and the second electric line 124 are disposed. Furthermore, according to an embodiment, the stem 126 may be configured to facilitate passage of gases to a rear surface of the wafer 112. Furthermore, according to an embodiment, for a precise temperature control of the wafer 112, a cooling gas, such as He gas, may be supplied between the electrostatic chuck 116 and the wafer 112.

A gate for entering and exiting the wafer 112 may be installed on a side wall of the reaction chamber 104. The wafer 112 may be loaded onto and unloaded from the wafer stage 114 through the gate.

The exhaust part 160 may be coupled to an exhaust port 161 that is installed at a lower portion of the reaction chamber 104, through an exhaust pipe. For example, the exhaust part 160 may include a vacuum pump, such as a turbo molecular pump, and may control a pressure of a processing space in the reaction chamber 104 to a desired vacuum level. Furthermore, for example, a second throttle valve 162 may be additionally installed in the exhaust port 161 to control a flow rate of a fluid that flows to the exhaust part 160. Furthermore, the exhaust part 160 may discharge process by-products and residual process gases that are generated in the reaction chamber 104, through the exhaust port 161.

In an embodiment, the plasma processing apparatus 100 may further include a coil 128, a plasma generation controller 132, a source gas supply part (i.e., source gas supplier) 136, and an equipment controller 150. Furthermore, in an embodiment, the plasma processing apparatus 100 may further include an additional gas supply part (i.e., additional gas supplier) 138.

The coil 128 may be disposed at a circumference of the remote plasma source 102. For example, the remote plasma source 102 may be implemented to have a dome-shaped outer wall, and the coil 128 may be disposed on the outer wall of the remote plasma source 102. However, this is provided as an example, and the remote plasma source 102 may be implemented in various forms, and the coil 128 may also be disposed around the remote plasma source 102 in various ways, including a direct-connection and/or an indirect-connection.

The plasma generation controller 132 may be electrically coupled to the coil 128 to generate plasma in the plasma area 130. For example, the plasma generation controller 132 may include a power supply for supplying electric power to the coil 128. For example, while plasma is generated, the plasma generation controller 132 may provide a specific electric power to the coil 128.

The source gas supply part 136 may be coupled to the remote plasma source 102 through a source gas supply line 135 to supply the source gas.

While the source gas supply part 136 supplies the source gas to the remote plasma source 102, ions and/or radicals may be generated in the plasma area 130. The ions generated in a plasma area 134, for example, may be filtered by an ion filter of the shower head 106. In this manner, the radicals generated in the plasma area 134 may be supplied to the wafer 112 in the reaction chamber 104 while limiting the ion impact.

In an embodiment, the source gas may include an oxygen-containing reactant, such as oxygen, or a nitrogen-containing reactant, such as nitrogen. Furthermore, in an embodiment, the source gas may include at least one of nitrogen gas, ammonia gas, or hydrogen gas. For example, the source gas supply part 136 may provide a source gas mixture including nitrogen gas, ammonia, and hydrogen gas to the remote plasma source 102, and nitrogen radicals, amine radicals, and hydrogen radicals may be generated in the plasma area 130. However, this is provided as an example, and the source gas according to embodiments is not limited thereto.

The additional gas supply part 138 may supply at least one additional gas to the remote plasma source 102. Accordingly, the source gas may be mixed with the additional gases. The additional gases may support or stabilize steady-state plasma conditions in the remote plasma source 102, or may assist ignition or extinguishment of the plasma.

In an embodiment, the additional gases may include helium (He), neon (Ne), argon (Ar), krypton (Kr), and xenon (Xe). Alternatively, in an embodiment, the additional gases may include hydrogen (H2) and ammonia (NH3). However, this is provided as an example, and the additional gases according to embodiments is not limited thereto.

In FIG. 1, it is illustrated that the source gas and the additional gases are provided to the remote plasma source 102 through different source gas supply lines 135 and additional gas supply lines 137, respectively. However, this is provided as an example, and according to an embodiment, the source gas and the additional gas may be provided to the remote plasma source 102 after being mixed in advance. For example the source gas provided from the source gas supply part 136 and the additional gas provided from the additional gas supply part 138 may be mixed before being introduced to the plasma area 130.

Plasma-activated gases, such as nitrogen radicals, amine radicals, and/or hydrogen radicals, may be provided from the remote plasma source 102 into the reaction chamber 104 through the shower head 106. Furthermore, some of the source gases may be provided from the remote plasma source 102 into the reaction chamber 104 through the shower head 106. In FIG. 1, for convenience of description, the source gases are indicated as “A” and “B”, and the plasma-activated gases are indicated as “C” and “D”.

In an embodiment, the shower head 106 may have a plurality of gas ports to diffuse the flow of plasma-activated gases into the reaction chamber 104. For example, the plurality of gas ports may be spaced apart from each other. The plurality of gas ports may smoothly disperse and diffuse the radicals (that is, plasma-activated gases) flowing from the remote plasma source 102 into the reaction area 110 of the reaction chamber 104.

The equipment controller 150 may control the overall operation of the semiconductor manufacturing equipment 1000. Furthermore, the equipment controller 150 may receive monitoring results from the reaction area monitoring apparatus 200, and may control related parameters and/or conditions based on the received monitoring results. The equipment controller 150 may include a memory 152 and a processor 154.

The memory 152 may store information that is necessary for the operation of the semiconductor manufacturing equipment 1000. The memory 152 may store instructions that are necessary for driving the semiconductor manufacturing equipment 1000. Furthermore, the memory 152 may operate as a working memory, in which instructions are executed. According to an embodiment, the memory 152 may be implemented to include at least one memory device.

The processor 154 may control the overall operation of the semiconductor manufacturing equipment 1000. The processor 154 may control conditions of the plasma processing apparatus 100 and/or the reaction area monitoring apparatus 200. According to an embodiment, the processor 154 may be implemented to include at least one processing circuitry. Furthermore, according to an embodiment, the processor 154 may be implemented as a single processor chip, or implemented as a plurality of multi-processors, or implemented as a multi-core processor. For example, when the processor 154 is implemented as a multi-processor, the functions of the processor 154, which will be described below, may be performed by different processors.

The processor 154 may control various parameters and/or conditions of the semiconductor manufacturing equipment 1000. In particular, the processor 154 according to an embodiment may receive real-time monitoring results from the reaction area monitoring apparatus 200, and may control parameters and/or conditions of the related process or equipment to implement desired performance and process results based on the received monitoring results.

In an embodiment, the processor 154 may communicate with the plasma generation controller 132 to control plasma parameters and/or conditions at the remote plasma source 102. Based on real-time monitoring results received from the reaction area monitoring apparatus 200, the processor 154 may control parameters and/or conditions at the remote plasma source 102 to optimize the generation of target radicals. For example, the parameters and/or conditions at the remote plasma source 102 may include a recipe parameter that is provided to the remote plasma source 102 and a radio frequency (RF) power that is supplied to the coil 128.

In an embodiment, the processor 154 may communicate with the source gas supply part 136 to control parameters and/or conditions in the source gas supply part 136. Based on real-time monitoring results received from the reaction area monitoring apparatus 200, the processor 154 may control parameters and/or conditions in the source gas supply part 136. For example, the parameters and/or conditions of the source gas supply part 136 may include a flow rate of the source gas, a flow rate ratio, and the like.

In an embodiment, the processor 154 may communicate with the additional gas supply part 138 to control parameters and/or conditions in the additional gas supply part 138. Based on real-time monitoring results received from the reaction area monitoring apparatus 200, the processor 154 may control parameters and/or conditions in the additional gas supply part 138. For example, parameters and/or conditions in the additional gas supply part 138 may include a flow rate of the additional gas, a flow rate ratio, and the like.

In an embodiment, the processor 154 may communicate with the wafer stage 114 to control parameters and/or conditions in the wafer stage 114. Based on real-time monitoring results received from the reaction area monitoring apparatus 200, the processor 154 may control parameters and/or conditions in the wafer stage 114. For example, the parameters and/or conditions in the wafer stage 114 may include up/down movement of the wafer stage 114, electrostatic chucking and dechucking, temperature, and the like.

Furthermore, based on the real-time monitoring results received from the reaction area monitoring apparatus 200, the processor 154 may control parameters and/or conditions, such as a pressure in the reaction chamber 104, a pressure in the remote plasma source 102, a temperature, a process time, an idle time, a retention time, a schedule parameter, RF power setting, frequency setting, a duty cycle, a pulse time, and the like, so that desired performance and process results may be implemented.

In an embodiment, the equipment controller 150 may be a component of the plasma processing apparatus 100 and may be electrically coupled to the reaction area monitoring apparatus 200. Alternatively, in an embodiment, the equipment controller 150 may be a component of the reaction area monitoring apparatus 200, and may be electrically coupled to the plasma processing apparatus 100. Alternatively, in an embodiment, the equipment controller 150 may be provided independently, and may be electrically coupled to the plasma processing apparatus 100 and the reaction area monitoring apparatus 200. Alternatively, in an embodiment, the equipment controller 150 may be an entirety or a part of a host computer system, or may be coupled to the host computer system through a network. For example, the equipment controller 150 may be an entirety or part of a fab host computer system that may enable remote access of the wafer processing operations. The equipment controller 150 may monitor semiconductor manufacturing operations, examine a history of past manufacturing operations, examine trends or performance metrics from semiconductor manufacturing operations, change parameters and/or conditions, or set processing stages.

Continuing with reference to FIG. 1, the reaction area monitoring apparatus 200 may include an analyzer 210 and at least one valve for coupling the analyzer 210 to the plasma processing apparatus 100. In an embodiment, the reaction area monitoring apparatus 200 may include an analyzer 210, a manual valve 220 and 260, a needle valve 230, an on-off valve 240, a pipeline 282 and 284, a first heating jacket 292, and a second heating jacket 294.

The analyzer 210 may receive a gas mixture of the reaction area 110 in the reaction chamber 104 through a first pipeline 282. For example, the gas mixture in the reaction chamber 104 may be provided to the analyzer 210 through a sampling inlet 105 and the first pipeline 282. Here, the gas mixture may include a gas before reaction with the wafer 112 and/or a gas after reaction with the wafer 112 in the reaction area 110.

For convenience of description, in the specification, it is assumed that the gas indicated as “A” (hereinafter, “gas A”) and the gas indicated as “B” (hereinafter, “gas B”) are source gases. It is assumed that the gas indicated as “C” (hereinafter, “gas C”) is a plasma-activated gas, and is an etch component. It is assumed that the gas indicated as “D” (hereinafter, “D gas”) is a plasma-activated gas and is a passivation component.

In this case, a plasma-activated gas including an etch component (e.g., gas “C”) and/or a passivation component (e.g., gas “D”) may be generated through plasma discharge for the source gas (e.g., gas “A” and/or gas “B”). Thereafter, a gas mixture including gas “A” and gas “B” that are the source gases, gas “C” that is the etch component, and gas “D” that is the passivation component may be provided to the reaction chamber 104. Thereafter, gas “C” that is the etch component and/or gas “D” that is the passivation component may react with the wafer 112. Accordingly, gas “A”, gas “B”, gas “C”, gas “D”, and other by-product gases after the reaction may exist in the reaction area 110. The gas mixture in the reaction area 110 may be provided to the analyzer 210 through the sampling inlet 105 and the first pipeline 282.

The analyzer 210 may receive the gas mixture through the first pipeline 282, and may monitor it in real time. Furthermore, the analyzer 210 may be coupled to the exhaust part 160 through a second pipeline 284, and may discharge the gas mixture through the second pipeline 284.

The analyzer 210 may perform a monitoring operation and/or an analysis operation for the gas mixture. For example, the analyzer 210 may monitor and analyze an intensity signal for each of the gases included in the gas mixture in real time. Alternatively, for example, the analyzer 210 may monitor and analyze the amount, concentration, composition, and/or ratio of the etch component and/or the passivation component included in the gas mixture.

In an embodiment, the analyzer 210 may be implemented to include a time-of-flight mass spectrometer (TOF-MS). In this case, the analyzer 210 may perform a mass analysis of the gas in the reaction area 110 in the plasma processing apparatus 100 in real time through the pipeline 282 and 284, and may output spectrum data for each gas component. For example, the gas in the reaction area 110 may be introduced to the analyzer 210 through the first pipeline 282, and discharged through the second pipeline 284. Accordingly, the analyzer 210 implemented with TOF-MS may perform real-time monitoring and analysis operations for unknown chemistry components that are not exactly known to be generated after plasma discharge. Furthermore, the analyzer 210 implemented with TOF-MS may accurately process many gases, and may support a fast data processing speed and a high resolution.

However, this is an example, and embodiments are not limited thereto. According to an embodiment, the analyzer 210 may be implemented as an analyzer using mass spectrometry, such as a residual gas analyzer (RGA), a quadruple mass analyzer (QMS). Alternatively, according to an embodiment, the analyzer 210 may be implemented by using gas chromatography, or may be implemented by using spectrometry.

Manual valves 220 and 260 may be provided to mount and demount the reaction area monitoring apparatus 200 on and from the plasma processing apparatus 100. For example, the first manual valve 220 may correspond to a sampling inlet 105 of the plasma processing apparatus 100, and the second manual valve 260 may correspond to a sampling outlet 164 of the plasma processing apparatus 100. The first and second manual valves 220 and 260 may mount and demount the reaction area monitoring apparatus 200 on and from the plasma processing apparatus 100 without breaking the vacuum state of the plasma processing apparatus 100.

Each of the needle valve 230 and a throttle valve 250 may control the flow rate of the reaction area monitoring apparatus 200. Accordingly, an optimum pressure state may be maintained for the monitoring operation and/or the analysis operation of the analyzer 210.

For example, the needle valve 230 is a type of a flow rate control valve, and may be located at a front end of the analyzer 210. The needle valve 230 may control a flow rate in the first pipeline 282 so that an optimum pressure state required by the analyzer 210 may be maintained.

For example, the throttle valve 250 is a type of a flow rate control valve, and may be located at a rear end of the analyzer 210. The throttle valve 250 may control a flow rate in the second pipeline 284 so that an optimum pressure state required by the analyzer 210 may be maintained.

However, this is an example, and the flow rate control valve of the reaction area monitoring apparatus 200 may be provided in various ways. For example, the reaction area monitoring apparatus 200 may include at least one flow rate control valve, and the at least one flow rate control valve may be located at a front end of the analyzer 210, a rear end of the analyzer 210, or both the front end and the rear end of the analyzer 210.

The on-off valve 240 may allow or block introduction of gas into the reaction area monitoring apparatus 200 through an on-off operation. For example, the on-off valve 240 may be located in the first pipeline 282, and may selectively block the flow of the gas in the first pipeline 282. However, this is provided as an example, and according to an embodiment, the on-off valve 240 may be located in the second pipeline 284, or may be located in both the first pipeline 282 and the second pipeline 284.

In an embodiment, the on-off valve 240 may be turned on and off in conjunction with in-out information of the wafer 112 and/or a lot.

For example, when the wafer 112 is loaded into the plasma processing apparatus 100, the on-off valve 240 may be turned on, and thus the gas mixture may be provided to the analyzer 210. For example, when the wafer 112 is unloaded from the plasma processing apparatus 100, the on-off valve 240 may be turned off, and thus the gas mixture may be blocked from being provided to the analyzer 210.

In an embodiment, the on-off valve 240 may be operated in synchronization with a power-on or power-off operation of the analyzer 210.

For example, when the wafer 112 is loaded into the plasma processing apparatus 100, the on-off valve 240 may be turned on and the analyzer 210 may be powered on. For example, when the wafer 112 is unloaded from the plasma processing apparatus 100, the on-off valve 240 may be turned off and the analyzer 210 may be powered off. In this case, the analyzer 210 may be powered on only when the plasma processing apparatus 100 is actually driven, and accordingly, a service life of the analyzer 210 may be extended compared to a case, in which it is always powered on. Furthermore, because the analyzer 210 is operated in synchronization when the plasma processing apparatus 100 is actually driven, the monitoring information and/or analysis information generated in the analyzer 210 may be efficiently used as fault detection classification (FDC) information.

A first heating jacket 292 and a second heating jacket 294 may be provided to prevent gas from solidifying into powder in the reaction area monitoring apparatus 200 and causing process defects. For example, the first heating jacket 292 and the second heating jacket 294 may be provided to apply heat to the pipeline 282 and 284 and/or the valve 220, 230, 240, 250, and 260 to maintain an internal temperature at a constant level to prevent by-products from being generated in the pipeline 282 and 284 and/or the valve 220, 230, 240, 250, and 260.

In an embodiment, the first heating jacket 292 may be provided to surround the first pipeline 282, and may prevent a passivation component and/or an etch component from being deposited in the first pipeline 282, and the second heating jacket 292 is provided to surround the second pipeline 284, and may prevent a passivation component and/or an etch component from being deposited in the second pipeline 284. Furthermore, a heating jacket may be also provided in an interior or an exterior of at least one of the valves 220, 230, 240, 250, and 260, and may prevent a passivation component and/or an etch component from being deposited in the valve 220, 230, 240, 250, and 260.

In an embodiment, the first heating jacket 282 and the second heating jacket 292 may be operated in conjunction with the on-off operation of the on-off valve 240 and/or the power on-off operation of the analyzer 210. That is, when the on-off valve 240 is turned on or the analyzer 210 is powered on, the first heating jacket 282 and the second heating jacket 292 may also provide heat to the corresponding pipeline or valve. Accordingly, the power consumption for driving the first heating jacket 282 and the second heating jacket 292 may be minimized.

As described above, the semiconductor manufacturing equipment 1000 according to an embodiment includes a plasma processing apparatus 100 and a reaction area monitoring apparatus 200, and the reaction area monitoring apparatus 200 may support a real-time monitoring operation for the reaction area 110 in the plasma processing apparatus 100, and the plasma processing apparatus 100 may control related parameters to implement required performance and process results based on the monitoring results. Accordingly, the semiconductor manufacturing equipment 1000 may efficiently find optimal process and facility conditions for implementing required performance and process results.

The desired performance or process results described in FIG. 1 may include items generally used for verifying a process performance and verifying a yield performance, such as an etch amount, a passivation layer amount, a deposition amount, uniformity, selectivity, an open CD, CD loading, by-products (residues), and pitting/shorts.

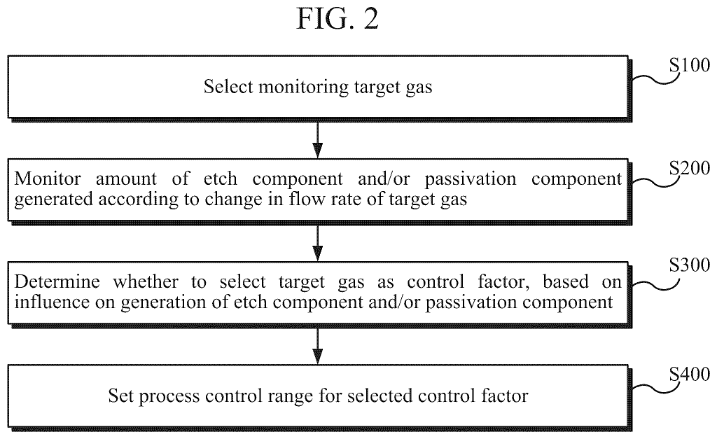

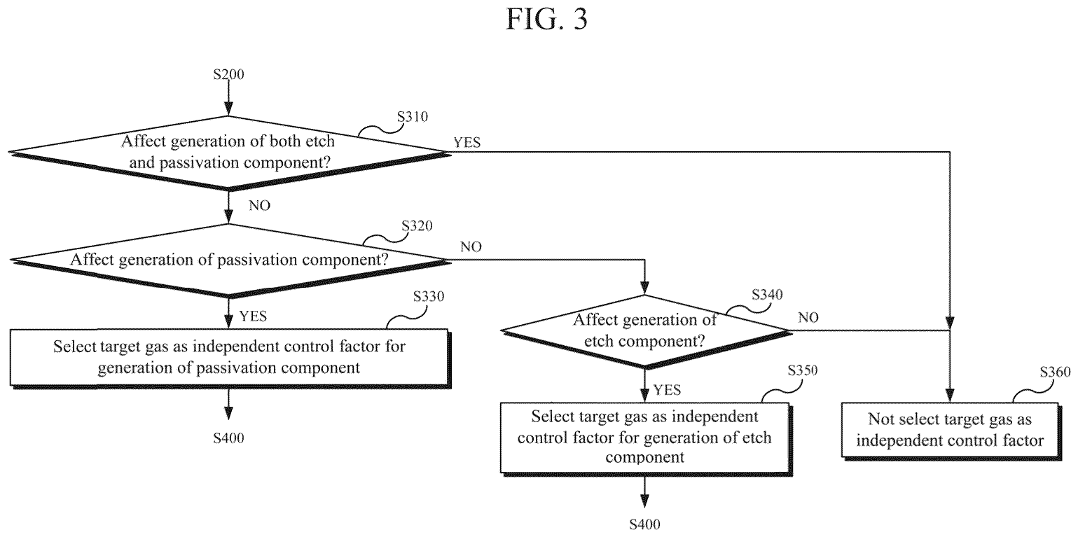

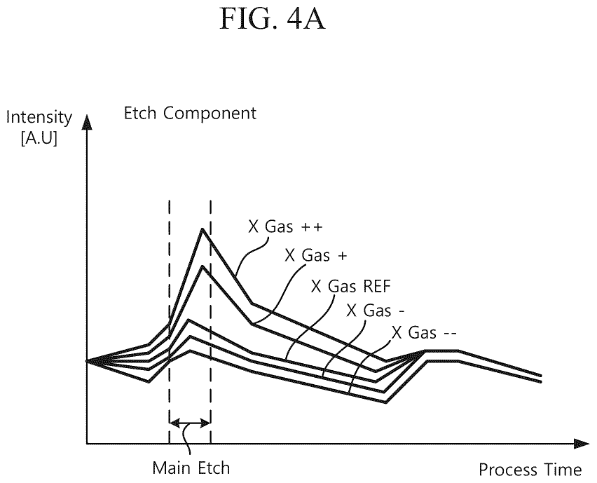

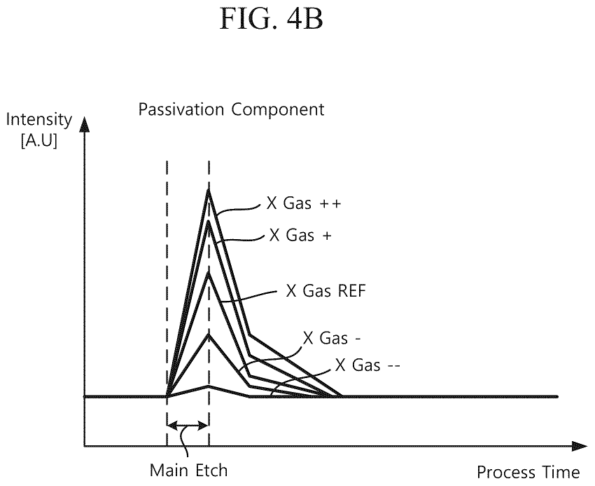

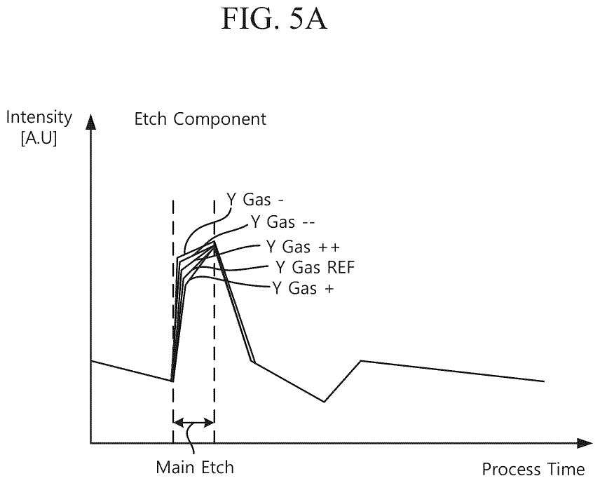

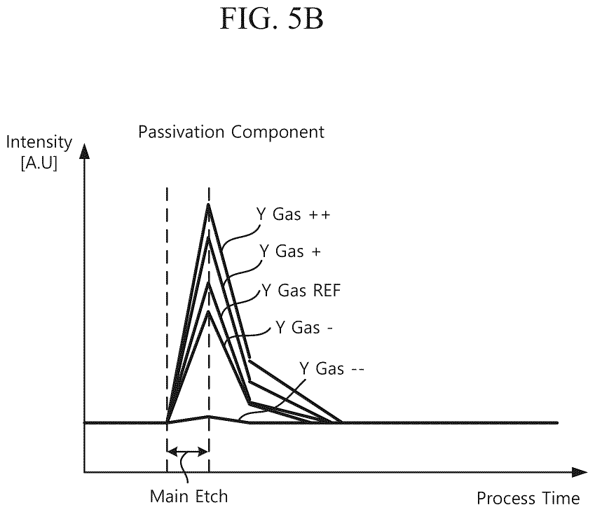

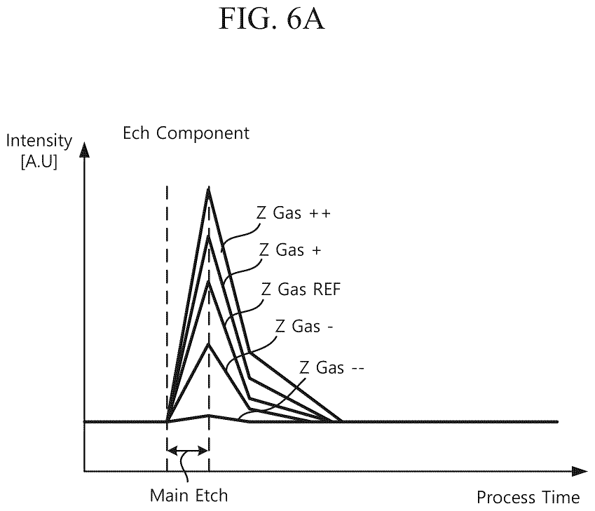

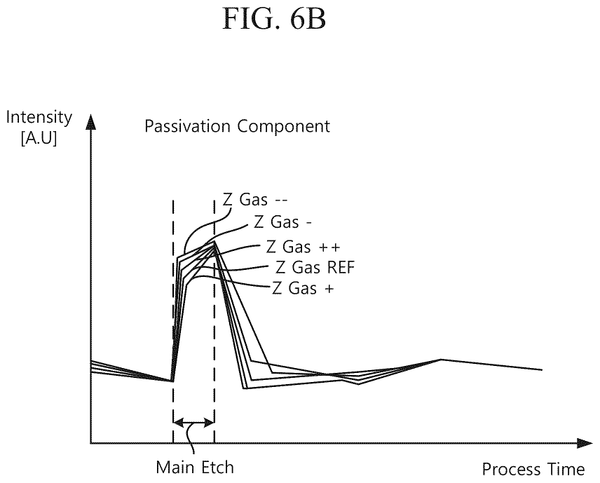

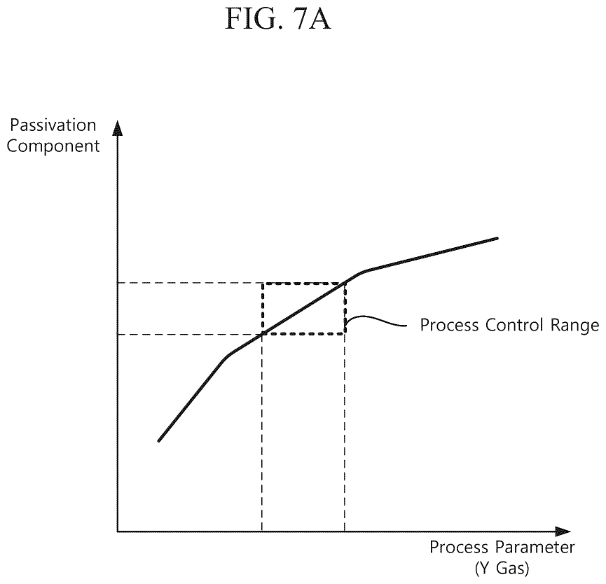

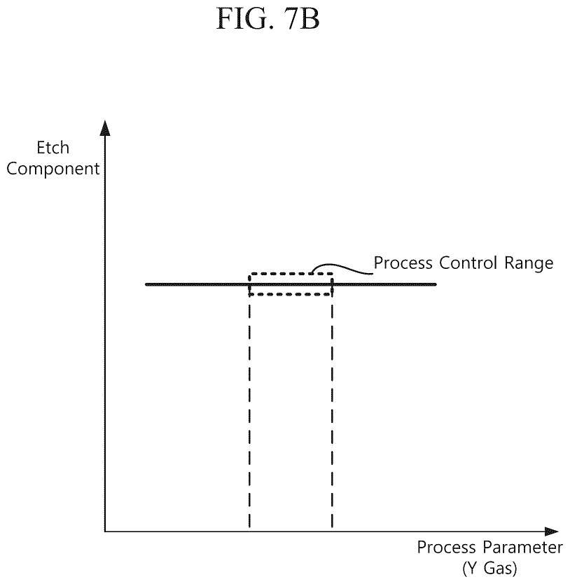

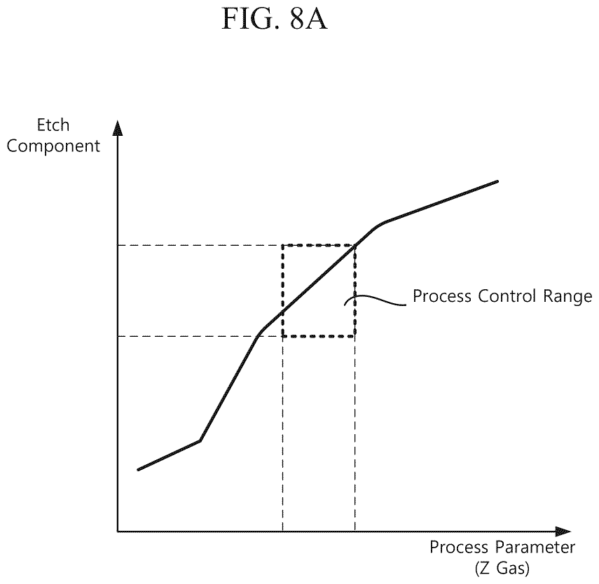

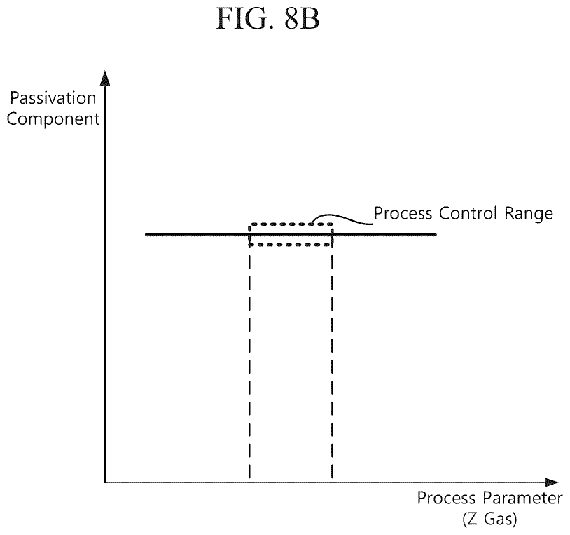

FIGS. 2 to 8B are views illustrating the operation of semiconductor manufacturing equipment 1000 according to an embodiment. In detail, FIG. 2 illustrates a flowchart illustrating an example of a real-time monitoring operation for a reaction area and a control factor setting operation. FIG. 3 illustrates a flowchart illustrating an example of an operation for selecting a control factor. In FIGS. 4A and 4B, an example of the monitoring results for an etch component and a passivation component when gas “X” is introduced as a source gas is illustrated by way of example. In FIGS. 5A and 5B, an example of the monitoring results for an etch component and a passivation component when gas “Y” is introduced as a source gas is illustrated by way of example. In FIGS. 6A and 6B, an example of the monitoring results for an etch component and a passivation component when gas “Z” is introduced as a source gas is illustrated by way of example. In FIGS. 7A and 7B, an example of setting a control range when gas “Y” is selected as a control factor is illustrated. In FIG. 8A and FIG. 8B, an example of setting a control ranges when gas “Z” is selected as a control factor is illustrated.

First, referring to FIG. 2, in operation S100, a monitoring target gas may be selected.

For example, as illustrated in FIGS. 4A to 8B, the analyzer 210 of the reaction area monitoring apparatus 200 may select gas “X”, gas “Y”, and gas “Z”, among the source gases, as target gases that are to be monitored, respectively.

In operation S200, an amount of the etch component and/or the passivation component generated according to a change in a flow rate of the target gas may be monitored.

For example, as illustrated in FIGS. 4A to 8B, the analyzer 210 of the reaction area monitoring apparatus 200 may monitor the amount of the etch component and/or the passivation component generated according to the change in the flow rate of each of gas “X”, gas “Y”, and gas “Z”.

In operation S300, it may be determined whether to select the target gas as a control factor, based on an influence on the generation of the etch component and/or the passivation component.

For example, the processor 154 of the equipment controller 150 may receive real-time monitoring results from the reaction area monitoring apparatus 200, and determine whether the target gas has affected the generation of the etch component and/or the passivation component. Based on the determining, the processor 154 may select the target gas as an independent control factor of the process or equipment.

Referring to FIG. 3 for a more detailed description, in operation S310, it may be determined whether the target gas affected the generation of both the etch component and the passivation component.

For example, the processor 154 may receive real-time monitoring results from the reaction area monitoring apparatus 200, and may determine whether the target gas affected the generation of the etch component and/or the passivation component.

When the target gas affected the generation of both the etch component and the passivation component, the target gas may not be selected as an independent control factor (operation S360).

For example, referring to FIG. 4A and FIG. 4B, the monitoring results for the intensity of the etch component and the passivation component depending on the amount of gas “X” that which is a source gas, are illustrated by way of example. Here, “X Gas REF” indicates a reference amount of gas “X”, “X Gas +” indicates that gas “X” is introduced more than the reference amount, and “X Gas ++” indicates that gas “X” is introduced more than “X Gas +”. “X Gas−” indicates that gas “X” is introduced less than the reference amount, and “X Gas−−” indicates that gas “X” is introduced less than “X Gas−”.

For example, as illustrated in FIGS. 4A and 4B, in a main etch section, it may be identified that the amount of gas “X” introduced affects not only the generation of the etch component but also the generation of the passivation component. In this case, the processor 154 may determine that gas “X” is inappropriate as an independent control factor.

When the target gas does not affect the generation of both the etch component and the passivation component, operation S320 may be performed.

In operation S320, it may be determined whether the target gas affected the generation of the passivation component.

When the target gas affected the generation of the passivation component, the target gas may be selected as an independent control factor for the generation of the passivation component (operation S330).

For example, referring to FIG. 5A and FIG. 5B, the monitoring results for the intensity of the etch component and the passivation component depending on the amount of gas “Y” that which is a source gas, are illustrated by way of example. Here, “Y Gas REF” represents a reference input amount of gas “Y”, and “Y Gas +”, “Y Gas ++”, “Y Gas−”, and “Y Gas−−” represent input amounts of gas “Y” compared to the reference input amount.

For example, as illustrated in FIG. 5A, in the main etch section, an input amount of gas “Y” did not affect the generation of the etch component or had little effect. In contrast, as illustrated in FIG. 5B, it may be identified that an amount of gas “Y” introduced affected the generation of the passivation component. In this case, the processor 154 may select gas “Y” as an independent control factor for the passivation component.

When the target gas does not affect the generation of the passivation component, operation S340 may be performed.

In operation S340, it may be determined whether the target gas affected the generation of the etch component.

When the target gas affected the generation of the etch component, the target gas may be selected as an independent control factor for the generation of the etch component (operation S350).

For example, referring to FIG. 6A and FIG. 6B, the monitoring results for the intensity of the etch component and the passivation component depending on the amount of gas “Z” that which is a source gas, are illustrated by way of example. Here, “Z Gas REF” represents a reference input amount of gas “Z”, and “Z Gas +”, “Z Gas ++”, “Z Gas−”, and “Z Gas−−” represent input amounts of gas “Z” compared to the reference input amount.

For example, as illustrated in FIGS. 6A and 6B, in a main etch section, an input amount of gas “Z” affected the generation of the etch component, but did not affect or had little effect on the generation of the passivation component. In this case, the processor 154 may select gas “Z” as an independent control factor for the etch component.

When the target gas did not affect the generation of the etch component, it is determined that the target gas will not affect the generation of both the passivation component and the etch component, and thus may not be selected as an independent control factor (operation S360).

Referring back to FIG. 2, in operation S400, a process control range for the selected control factor may be set.

For example, as illustrated in FIGS. 7A and 7B, when gas “Y” is selected as an independent control factor for the passivation component, the processor 154 may analyze a production amount of the passivation component relative to the input amount of gas “Y”. Then, the processor 154 may set a section, in which an increase in the passivation component relative to the input amount of gas “Y” is linear, as a process control range.

Similarly, as illustrated in FIGS. 8A and 8B, when gas “Z” is selected as an independent control factor for the etch component, the processor 154 may analyze the production amount of the etch component relative to the input amount of gas “Z”. Thereafter, the processor 154 may set a section, in which an increase in the etch component relative to the input amount of gas “Z” is linear, as a process control range.

In this way, the semiconductor manufacturing equipment 1000 according to an embodiment may support real-time monitoring operation for the reaction area 110 in the reaction chamber 104 and control related parameters to implement required performance and process results based on the monitoring results. Accordingly, the semiconductor manufacturing equipment 1000 may efficiently find optimal process and facility conditions for implementing required performance and process results.

The above description is provided as an example, and it will be understood that the technical idea is not limited thereto and may be variously modified. Hereinafter, various modifications according to the technical idea will be described in more detail.

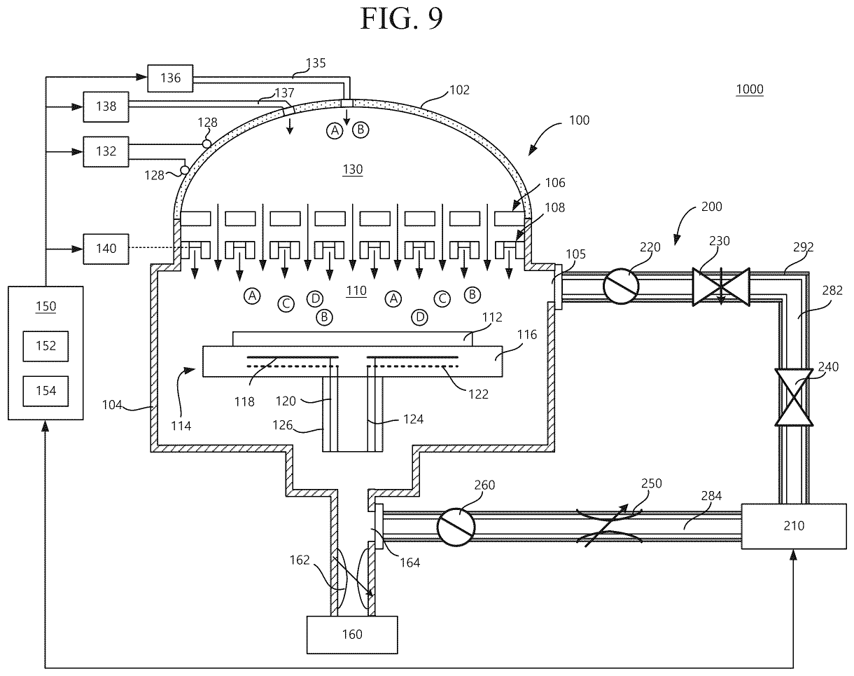

FIG. 9 is a view illustrating semiconductor manufacturing equipment according to an embodiment. The semiconductor manufacturing equipment of FIG. 9 is similar to the semiconductor manufacturing equipment of FIG. 1. Accordingly, identical or similar components are indicated by identical or similar reference numerals, and a repeated description thereof will be omitted below.

Compared to the semiconductor manufacturing equipment of FIG. 1, the semiconductor manufacturing equipment 1000 of FIG. 9 may further include a precursor supply source 140 and gas outlets 108.

The precursor supply source 140 may be configured to provide a precursor according to a control of the equipment controller 150, and the gas outlets 108 may be configured to provide a precursor received from the precursor supply source 140 into the reaction chamber 104. For example, the precursor may include a silicon-containing precursor, such as SiCl₄, SiHCl₃, or the like.

In an embodiment, the gas outlets 108 may include a plurality of openings that are spaced apart from each other. In an embodiment, the gas outlets 108 may be located below the shower head 106. Alternatively, in an embodiment, the gas outlets 108 may be implemented as a part of the shower head 106. In this way, precursors may be provided into the reaction chamber 104 without being exposed to plasma in the remote plasma source 102.

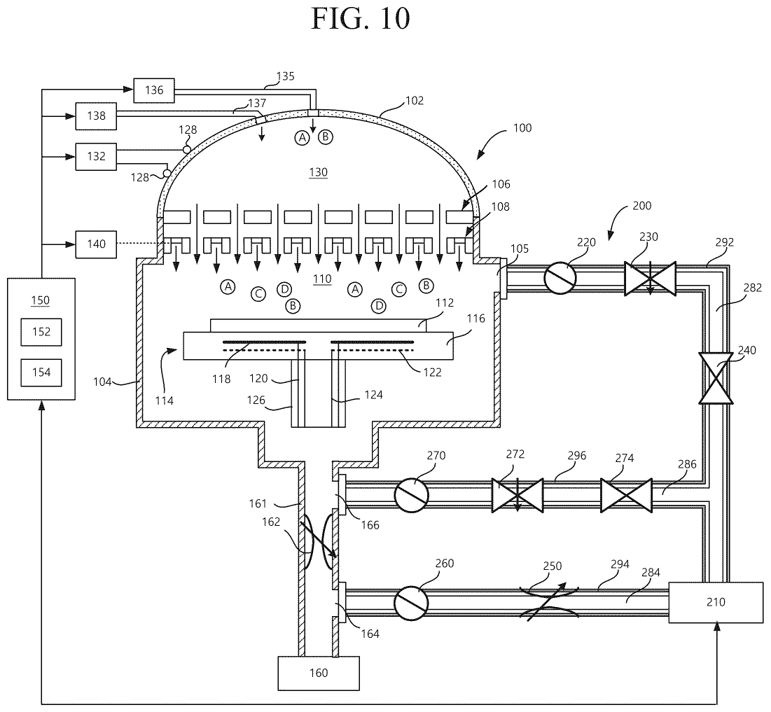

FIG. 10 is a view illustrating semiconductor manufacturing equipment according to an embodiment.

The semiconductor manufacturing equipment of FIG. 10 is similar to the semiconductor manufacturing equipment of FIG. 1 and FIG. 9. Accordingly, identical or similar components are indicated by identical or similar reference numerals, and a repeated description thereof will be omitted below.

Compared to the semiconductor manufacturing equipment of FIG. 1 and FIG. 9, the semiconductor manufacturing equipment of FIG. 10 may not only monitor the gas mixture in the reaction area 110 in the reaction chamber 104 in real time, but also monitor the gas mixture at an exhaust end in real time. To achieve this, the reaction area monitoring apparatus 200 may further include a third pipeline 286, a third manual valve 270, a second needle valve 272, a second on-off valve 274, and a third heating jacket 296 for monitoring gas mixture at the exhaust end.

The third pipeline 286 may be coupled to the second sampling inlet 166 and the analyzer 210, and may lead the gas mixture of the exhaust port 161 to the analyzer 210.

The third manual valve 270 may be configured to mount and demount the reaction area monitoring apparatus 200 on and from the exhaust port 161 of the plasma processing apparatus 100.

The second needle valve 272 is a type of a flow rate control valve, and may be located at a front end of the analyzer 210. The second needle valve 272 may control a flow rate in the third pipeline 286 so that an optimum pressure state required by the analyzer 210 may be maintained.

The second on-off valve 274 may allow or block introduction of gas into the reaction area monitoring apparatus 200 through an on-off operation. For example, the second on-off valve 274 may be located in the third pipeline 286, and may selectively block the flow of the gas in the third pipeline 286.

In an embodiment, the second on-off valve 274 may be turned on and off in conjunction with in-out information of the wafer 112 and/or a lot. Furthermore, in an embodiment, the second on-off valve 274 may be operated in synchronization with a power-on or power-off operation of the analyzer 210. For example, when the wafer 112 is loaded into the plasma processing apparatus 100, the second on-off valve 274 may be turned on and the analyzer 210 may be powered on. For example, when the wafer 112 is unloaded from the plasma processing apparatus 100, the second on-off valve 274 may be turned off and the analyzer 210 may be powered off.

In an embodiment, the reaction area monitoring apparatus 200 may selectively perform a monitoring operation for any one of the gas mixture in the reaction area 110 or the gas mixture in the exhaust port 161 in the reaction chamber 104. In this regard, the monitoring operation for the gas mixture in the reaction area 110 and the monitoring operation for the gas mixture in the exhaust port 161 may be performed respectively, and accordingly, a chemical analysis may be performed through a change in a gas partial pressure before and after the actual reaction.

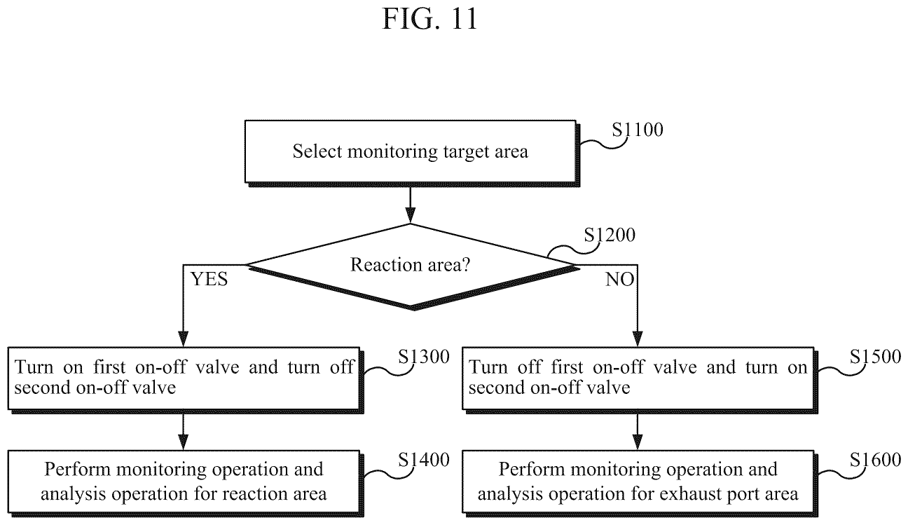

FIG. 11 illustrates an example of operation of the semiconductor manufacturing equipment of FIG. 10, for selecting a monitoring target area.

In operation S1100, a monitoring target area may be selected.

For example, the processor 154 of the equipment controller 150 may select an area that is to be monitored, among the areas corresponding to the reaction area 110 or the exhaust port 161 in the reaction chamber 104.

In operation S1200, it may be determined whether the monitoring target area is the reaction area 110 or an area corresponding to the exhaust port 161.

When the monitoring target area is the reaction area 110, operations S1300 and S1400 may be performed.

In operation S1300, the first on-off valve 240 may be turned on and the second on-off valve 274 may be turned off. Accordingly, the gas mixture in the reaction area 110 may be provided to the analyzer 210. According to an embodiment, the first needle valve 230 may control the flow rate in the first pipeline 282 to satisfy the optimum pressure condition required by the analyzer 210.

In operation S1400, a monitoring operation and an analysis operation for the reaction area may be performed. For example, the analyzer 210 may receive the gas mixture in the reaction area 110 in the reaction chamber 104 through the first sampling inlet 105 and the first pipeline 282, and may perform a real-time monitoring operation for the received gas mixture. Thereafter, the analyzer 210 may discharge the gas mixture through the second pipeline 284 coupled to a sampling outlet 164. In this case, the power-on or power-off operation of the analyzer 210 may be performed in synchronization with the turn-on or turn-off operation of the first on-off valve 240.

When the monitoring target area is not the reaction area 110, that is, in the area corresponding to the exhaust port 161, operations S1500 and S1600 may be performed.

In operation S1500, the first on-off valve 240 may be turned off, and the second on-off valve 274 may be turned on. Accordingly, the gas mixture in the area corresponding to the exhaust port 161 may be provided to the analyzer 210. According to an embodiment, the second needle valve 272 may control the flow rate of the third pipeline 286 to satisfy the optimum pressure condition required by the analyzer 210.

In operation S1600, a monitoring operation and an analysis operation for the area corresponding to the exhaust port 161 may be performed. For example, the analyzer 210 may receive the gas mixture of the area corresponding to the exhaust port 161 through the second sampling inlet 166 and the third pipeline 286, and may perform a real-time monitoring operation for the received gas mixture. Thereafter, the analyzer 210 may discharge the gas mixture through the second pipeline 284 coupled to a sampling outlet 164. In this case, the power-on or power-off operation of the analyzer 210 may be performed in synchronization with the turn-on or turn-off operation of the second on-off valve 274.

In this way, in an embodiment, the reaction area monitoring apparatus 200 may selectively perform a monitoring operation for any one of the gas mixture in the reaction area 110 or the gas mixture in the exhaust port 161 in the reaction chamber 104.

However, this is an example, and embodiments are not limited thereto. According to an embodiment, the first on-off valve 240 and the second on-off valve 274 may be turned on simultaneously, in which case, the monitoring operation for the reaction area 110 and the monitoring operation for the area of the exhaust port 161 may be performed simultaneously. Furthermore, according to an embodiment, the reaction area monitoring apparatus 200 may be implemented to monitor only the area corresponding to the exhaust port 161. In this case, the reaction area monitoring apparatus 200 may not be provided with the first pipeline 282 and the valve corresponding thereto.

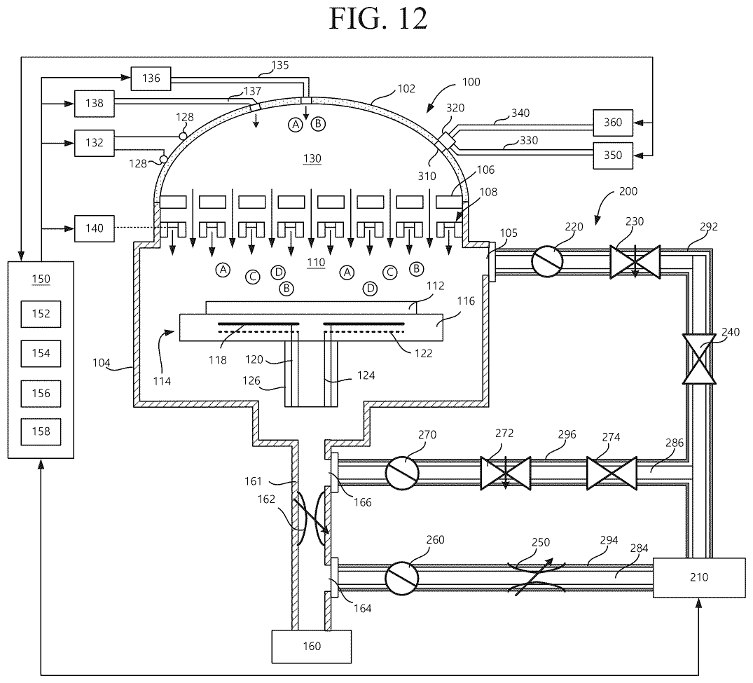

FIG. 12 is a view illustrating semiconductor manufacturing equipment according to an embodiment. The semiconductor manufacturing equipment of FIG. 12 is similar to the semiconductor manufacturing equipment of FIGS. 1, 9, and 10. Accordingly, identical or similar components are indicated by identical or similar reference numerals, and a repeated description thereof will be omitted below.

Compared to the semiconductor manufacturing equipment of FIG. 10, the semiconductor manufacturing equipment of FIG. 12 may be implemented to additionally monitor the remote plasma source 102. To achieve this, the semiconductor manufacturing equipment of FIG. 12 may further include a view port 310, a calibration adapter 320, a calibration apparatus 350, and a sensor 360.

The view port 310 may be provided on one side surface of the remote plasma source 102. The view port 310 may include, for example, quartz or sapphire. The view port 310 may include an optical filter that transmits only light of certain wavelengths.

A calibration adapter 320 may be provided on the view port 310. The calibration adapter 320 may be coupled to a calibration device 350 through a first optical fiber 330.

The calibration device 350 may include a light source and a series of optical systems for acquiring a reference spectrum of the light source. For example, the calibration device 350 may include a Hg—Ar light source. When there are a plurality of reaction chambers 104, in which an etching process is performed, a wavelength spectrum measured for the same material may be different in each of the remote plasma sources 102. For example, a wavelength spectrum measured from one remote plasma source 102 may exhibit a result that is shifted to a certain extent from a wavelength spectrum measured from another remote plasma source 102. The calibration device 350 may compensate for this wavelength shift so that an accurate wavelength is measured. That is, the calibration device 350 may match the wavelength spectrums of the different remote plasma sources 102 to each other.

The view port 310 may be coupled to the sensor 360 through the calibration adapter 320 and the second optical fiber 340. The sensor 360 may include, for example, an optical emission spectroscope (OES). The sensor 360 may convert at least a portion of an optical signal transmitted from the view port 310 into an electrical signal. According to an embodiment, a plurality of view ports 310, and a plurality of sensors 360 that detect optical signals that come out of the view ports 310 may be provided.

The calibration device 350 and the sensor 360 may be coupled to the equipment controller 150. The processor 154 of the equipment controller 150 may analyze the electrical signal from the sensor 360, and may generate a control signal according to the analysis result.

In this way, the semiconductor manufacturing equipment 1000 according to an embodiment may not only support a real-time monitoring operation for the reaction area 110 in the reaction chamber 104, but also support a monitoring operation for the plasma area 130 in the remote plasma source 102.

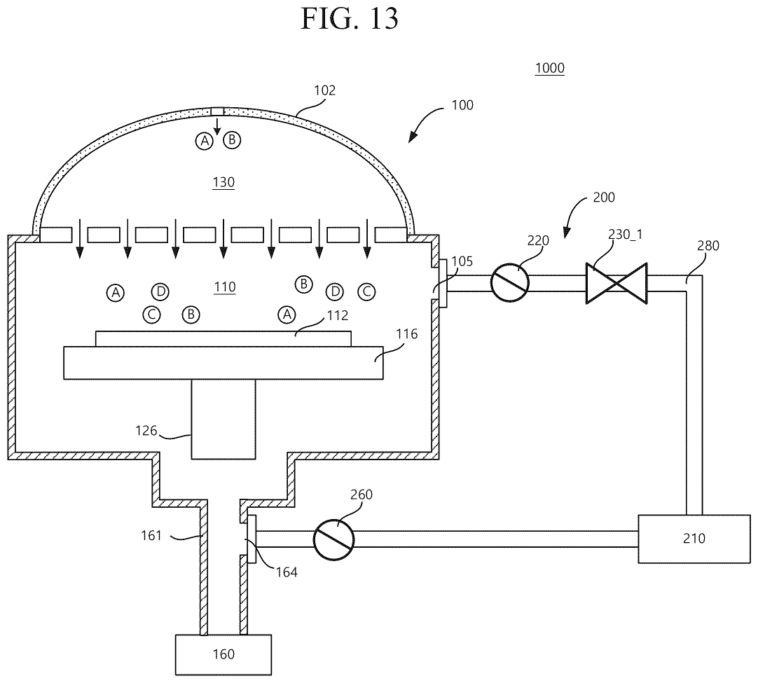

FIG. 13 is a view illustrating semiconductor manufacturing equipment according to an embodiment.

The semiconductor manufacturing equipment 1000 of FIG. 13 is similar to the semiconductor manufacturing equipment of FIG. 1. Accordingly, identical or similar components are indicated by identical or similar reference numerals, and a repeated description thereof will be omitted below.

With respect to FIG. 1, it has been described that the semiconductor manufacturing equipment 1000 includes an equipment controller 150. However, this is an example, and embodiments are not limited thereto. For example, as illustrated in FIG. 13, the semiconductor manufacturing equipment 1000 may not include an equipment controller. In this case, the equipment controller may be implemented as a part of a host system, implemented in the cloud, or implemented as a separate facility independent of the semiconductor manufacturing equipment 1000.

Furthermore, in FIG. 1, it has been described that the reaction area monitoring apparatus 200 includes an on-off valve, a needle valve, and a throttle valve. However, this is an example, and embodiments are not limited thereto. For example, as illustrated in FIG. 13, the reaction area monitoring apparatus 200 may be implemented to include pipelines 280, manual valves 220 and 260, and a single flow rate control valve 230_1. In this case, the flow rate control valve 230_1 may be a needle valve or a throttle valve.

The semiconductor manufacturing equipment according to an embodiment may support the real-time monitoring of the reaction area in the reaction chamber. Accordingly, the parameters and/or conditions for the optimum process or facility to implement the performance and process results required in the semiconductor manufacturing equipment may be efficiently found.

While aspects of embodiments have been particularly shown and described, it will be understood that various changes in form and details may be made therein without departing from the spirit and scope of the following claims.

What is claimed is:

1. Semiconductor manufacturing equipment comprising:
a remote plasma source used configured to generate plasma;
a reaction chamber coupled to the remote plasma source, and defining a space in which a wafer is processed;
an exhaust pipeline coupled to the reaction chamber through an exhaust port, and configured to discharge by-products generated in the reaction chamber;
a first pipeline coupled to the reaction chamber;
a second pipeline coupled to the exhaust pipeline through the exhaust port; and
an analyzer configured to analyze a first gas mixture received from the reaction chamber through the first pipeline.

2. The semiconductor manufacturing equipment of claim 1, further comprising a third pipeline coupled to the exhaust port,
wherein the analyzer is further configured to analyze a second gas mixture received from the exhaust port through the third pipeline.

3. The semiconductor manufacturing equipment of claim 2, further comprising:
a first on-off valve in the first pipeline, and configured to selectively allow or block flow of the first gas mixture to the analyzer; and
a second on-off valve in the third pipeline, and configured to selectively allow or block flow of the second gas mixture to the analyzer.

4. The semiconductor manufacturing equipment of claim 3, further comprising an equipment controller configured to:
based on a monitoring operation for the first gas mixture being performed, turn the first on-off valve on and the second on-off valve off; and
based on a monitoring operation for the second gas mixture being performed, turn the first on-off valve off, and the second on-off valve on.

5. The semiconductor manufacturing equipment of claim 4, wherein the equipment controller configured to:
based on the monitoring operation for the first gas mixture being performed, synchronize a power state of the analyzer with a state of the first on-off valve; and
based on the monitoring operation for the second gas mixture being performed, synchronize the power state of the analyzer with a state of the second on-off valve.

6. The semiconductor manufacturing equipment of claim 1, further comprising:
a view port located on a side surface of the remote plasma source;
a calibration adapter provided on the view port;
a calibration device coupled to the calibration adapter through a first optical fiber; and
a sensor coupled to the calibration adapter through a second optical fiber.

7. The semiconductor manufacturing equipment of claim 6, wherein the view port comprises an optical filter, and
wherein the sensor comprises an optical emission spectrometer.

8. The semiconductor manufacturing equipment of claim 1, further comprising:
a first manual valve configured to attach the first pipeline to the reaction chamber;
a second manual valve configured to attach the second pipeline to the exhaust port;
a needle valve in the first pipeline, wherein the needle valve is configured to control a flow rate of the first gas mixture flowing in the first pipeline;
a throttle valve in the second pipeline, wherein the throttle valve is configured to control a flow rate of the first gas mixture flowing in the second pipeline; and
an on-off valve in the first pipeline, wherein the on-off valve is configured to selectively allow flow of the first gas mixture to the analyzer.

9. The semiconductor manufacturing equipment of claim 8, further comprising:
a first heater configured to heat the first pipeline; and
a second heater configured to heat the second pipeline.

10. The semiconductor manufacturing equipment of claim 8, further comprising an equipment controller configured to, based on a monitoring operation for the first gas mixture being performed, synchronize a power state of the analyzer with a state of the on-off valve.

11. The semiconductor manufacturing equipment of claim 1, further comprising an equipment controller configured to control a parameter of the semiconductor manufacturing equipment based on a monitoring result for the first gas mixture.

12. The semiconductor manufacturing equipment of claim 11, wherein the equipment controller is further configured to identify whether a target gas in the first gas mixture is an independent control factor, based on a generated amount of an etch component or a generated amount of a passivation component according to a change in a flow rate of the target gas.

13. The semiconductor manufacturing equipment of claim 12, wherein the equipment controller is further configured to determine, based on the target gas affecting only generation of the passivation component, that the target gas is an independent control factor for the passivation component.

14. The semiconductor manufacturing equipment of claim 12, wherein the equipment controller is further configured to determine, based on the target gas affecting only generation of the etch component, that the target gas is an independent control factor for the etch component.

15. The semiconductor manufacturing equipment of claim 12, wherein the equipment controller is further configured to, based on the target gas affecting generation of both the passivation component and the etch component, not select the target gas as an independent control factor.

16. The semiconductor manufacturing equipment of claim 12, wherein the equipment controller is further configured to determines a section, in which the generated amount of the etch component or the generated amount of the passivation component is linear, as a processor control range for the target gas.

17. The semiconductor manufacturing equipment of claim 12, further comprising:
a plasma generation controller configured to control generation of plasma in the remote plasma source;
a source gas supplier configured to provide a source gas to the remote plasma source;
an additional gas supplier configured to provide an additional gas to the remote plasma source; and
a wafer stage in the reaction chamber, wherein the wafer stage is configured to support the wafer,
wherein the equipment controller is further configured to control at least one of a parameter for the plasma generation controller, a parameter for the source gas supplier, a parameter for the additional gas supplier, or a parameter for the wafer stage, based on the monitoring result.

18. The semiconductor manufacturing equipment of claim 1, further comprising a shower head located between the remote plasma source and the reaction chamber, wherein the shower head is configured to filter ions generated in the remote plasma source.

19. Semiconductor manufacturing equipment comprising:
a remote plasma source configured to generate plasma;
a reaction chamber coupled to the remote plasma source, and defining a space in which a wafer is processed;
a shower head located between the remote plasma source and the reaction chamber;
an exhaust pipeline coupled to the reaction chamber through an exhaust port, and configured to discharge by-products generated in the reaction chamber;
a first pipeline coupled to the reaction chamber;
a first manual valve configured to attach the first pipeline to the reaction chamber;
a needle valve in the first pipeline, wherein the needle valve is configured to control a flow rate of a gas mixture flowing in the first pipeline;
a second pipeline coupled to the exhaust pipeline through the exhaust port;
a second manual valve in the second pipeline, and used for mounting or demounting the second pipeline on or from the exhaust port;
a throttle valve in the second pipeline, wherein the throttle valve is configured to control a flow rate of the gas mixture flowing in the second pipeline;
an analyzer configured to analyze the gas mixture received from the reaction chamber through the first pipeline; and
an on-off valve in the first pipeline, wherein the on-off valve is configured to selectively allow flow of the gas mixture to the analyzer.

20. Semiconductor manufacturing equipment comprising:
a remote plasma source configured to generate plasma;
a reaction chamber coupled to the remote plasma source, and defining a space in which a wafer is processed;
an exhaust pipeline coupled to the reaction chamber through an exhaust port, wherein the exhaust pipeline is configured to discharge by-products generated in the reaction chamber;
an analyzer coupled to the reaction chamber through a first pipeline, wherein the analyzer is coupled to the exhaust pipeline through a second pipeline, and is configured to analyze a gas mixture received from the reaction chamber; and
an equipment controller configured to control at least one of a parameter for the remote plasma source or a parameter for the reaction chamber, based on a monitoring result received from the analyzer.
